(19) United States
(12) Patent Application Publication
(10) Pub. No.: US 2006/0084928 A1
(43) Pub. Date: Apr. 20, 2006

(54) Angiocath guide and stabilizer

(76) Inventor: Cayetano S. Munoz, El Mirage, AZ (US)

Correspondence Address: Cayetano S. Munoz, M.D., 12330 West Windrose Drive, El Mirage, AZ, 85335, US

(21) Appl. No.: 10/968,719

(22) Filed: Oct. 20, 2004

(51) Int. Cl. A61M 5/32

(52) U.S. Cl. 604/272

(57) ABSTRACT

A square foam ramp about the size of a quarter. On the mid line top side is a groove and a plastic tube about 22 F. which is slit on it's top side so as to accommodate a larger diameter angiocath needle. Sticking out from the bottom midline, a stiff straight nylon projects forward as a pointer. The bottom surface is provided with adhesive covered with a paper liner. Device is packed in sterile package.

### FIELD OF INVENTION

This is a simple medical device to facilitate insertion of an intravenous or arterial catheter (angiocath needle) utilized by medical personnel for transfusion of fluids intravenously or for monitoring arterial pressure and blood gas determination from extracted blood.

1. Background and Description of Prior Art

This is basically a thin small foam ramp with a groove and tube on the top wherein the angiocath is inserted toward the blood vessel (artery or vein). The bottom is provided with adhesive, covered with a paper liner. The bottom adheres to the skin. A stiff nylon hair like pointer projects from the bottom to line up forward with the vessel chosen for puncture.

2. Specifically Surveying Prior Art

U.S. Pat. No. 4,516,968—Catheter Shield and Method of Use

A catheter shield comprising of a dome with a radial slit to aid in placement around the catheter. This is anchored against the skin by the catheter and a tape strip.

U.S. Pat. No. 4,846,807—Intravenous Tubing Anchor and Shield

A continuous frame of resilient foam strapped to a patient (dorsum of the hand) with a central aperture of the frame surrounding the puncture area of an IV needle inserted into the patient. A separate dome is attached over the central aperture to shield the needle. The IV tube is anchored to the frame

U.S. Pat. No. 3,900,026—Device for Holding and Protecting Intravenous Injection Needle

It is a plastic transparent cap or cover with a peripheral skin contacting plunges, which is secured to the skin and protects the inserted needle. It may be self-adherent or tape secured.

U.S. Pat. No. 6,042,568—Patient Mounted Intravenous Protective Apparatus

A transparent tube with open sides which interlock and surround the forearm wherein the IV is inserted. Tube has foam at the front and at the end, in contact with skin for comfort. Tube locks in place.

U.S. Pat. No. 4,517,971—Guard for Venipuncture site and Catheter Retainer

A device to protect site and retain it. This is placed on top of IV site with the bottom part in contact with area around the needle. The top closes down and locks in place.

### Objective and Advantage

1. Simplicity; easy to manufacture; cheap and practical.

2. Less unnecessary hand motion while inserting needle; less pain.

3. Needle is secure and stable

4. Easy to master.

Variations:

It can be used in the insertion of arterial catheter for continuous blood pressure monitoring or blood gas analysis in the operating room, emergency rooms and intensive care units. Shape may vary. May consider a smaller front area [symbol] or may be circular or oblong.

[symbol]

### SUMMARY OF THE INVENTION

For Intravenous Catheters:

Place tourniquet proximal to puncture site (forearm, arm or leg) to distend vein. When desired puncture site is located, remove liner; place ramp with needle guide and pointer pointing towards vein directly in line. Press down to secure to skin. Insert angiocath through the proximal end of tube. Push down needle and make contact on puncture site. Push down till blood is withdrawn. Push plastic part of angiocath . . . Press down on the vein with a finger to stop blood flow while you remove the metal portion of the needle . . . connect tubing and secure with tape . . . Remove finger pressure.

For Arterial Catheters

After palpation of the pulse (radial or femoral), mark site. Place device with needle guide and ramp pointing down toward the pulsating site. Slowly insert angiocath through the proximal end of the tube. Push down and make contact on puncture site. Push down till blood is withdrawn. Slowly push down plastic part of angiocath. Apply pressure on artery with a finger while you extract metal part of needle and connect tubing. Flush tubing; secure with tape and bandage.

### BRIEF DESCRIPTION OF DRAWINGS

FIG. 1. Side view of angiocath guide and stabilizer.

FIG. 2. Back view of devise.

FIG. 3. Front view of devise.

FIG. 4. Oblique view of devise.

FIG. 5. Front view of devise.

FIG. 6. Top view of devise on dorsum of hand with tourniquet in place.

FIG. 7. View of devise on extended hand and radial artery site in place marked.

FIG. 8. Angiocath

### DETAILED DESCRIPTION OF PREFERRED EMBODIMENT

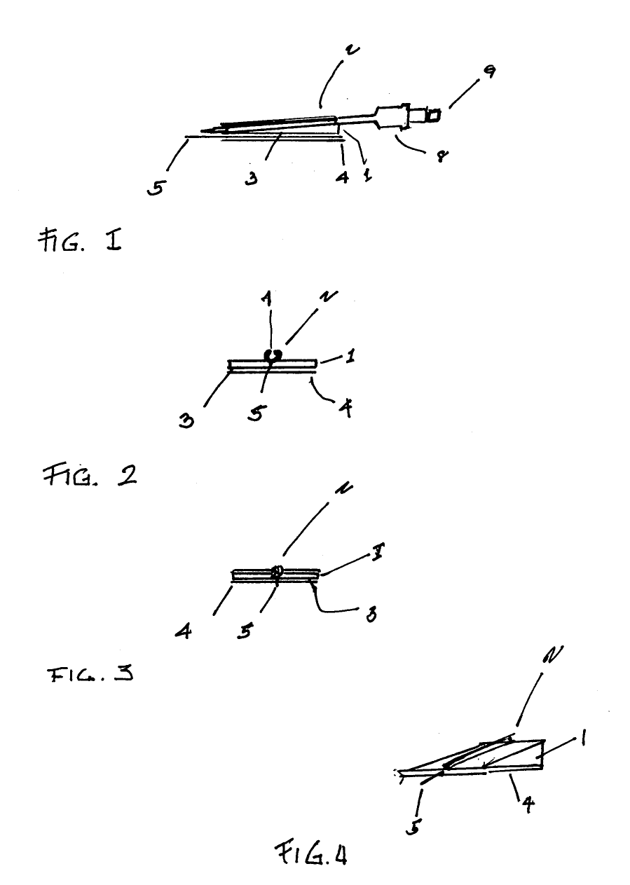

FIG. 1. Shows the component of the IV guide and stabilizer. It consists of a foam ramp (#1); square in shape, about the size or smaller than a quarter, with a groove and tube (#2). Tube has a slit on the top (#7) so it can permit a bigger needle through. Ramp has a sticky bottom (#3) covered by a removable paper liner (#4). Stiff nylon hairlike pointer projecting forward from the bottom (#5).

FIG. 2. Back view shows tube with a slit on top (#7); foam (#1); sticky base (#3); removable liner (#4).

FIG. 3. Front view.

FIG. 4. Oblique view shows slant to paper-thin front.

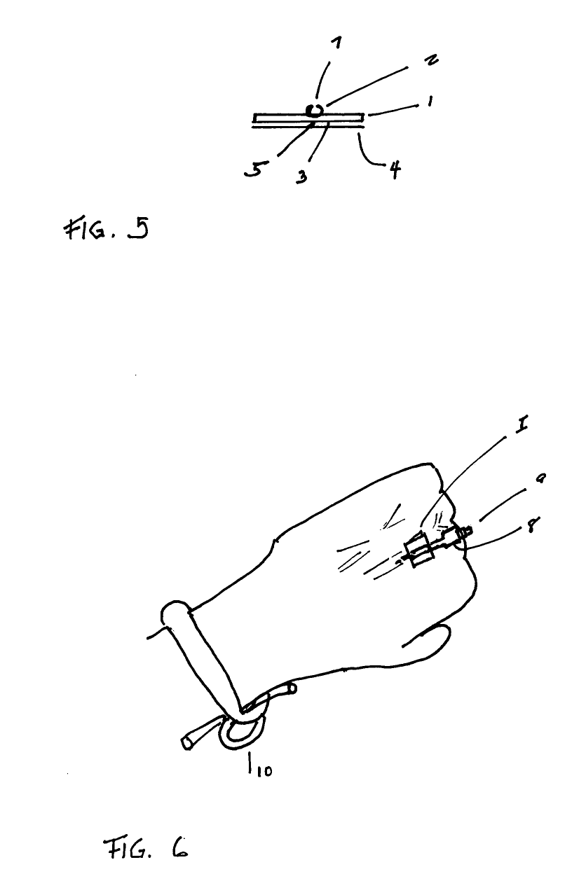

FIG. 5. View of tube with dorsal slit (#7).

FIG. 6. Shows forearm with catheter guide and stabilizer on desired site in line with vein.

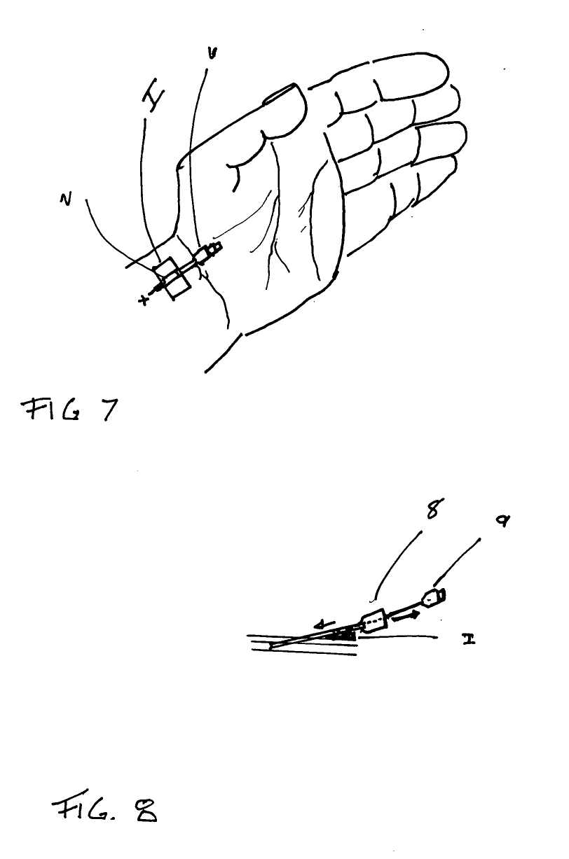

FIG. 7. Shows extended hand with guide and stabilizer in front of arterial pulsation site in line with artery. Needle pointing toward pulsation Angiocath (#6)

FIG. 8. Shows angiocath inserted into vessel (vein or artery). Plastic portion (#8); metal portion (#9); tourniquet (#10).

1. This is a simple devise to facilitate insertion of an angiocath into a vein or artery. It stabilizes the angiocath and insertion is smoother with less hand motion, so the patient feels less pain during puncture. It is cheap and easy to master.
